(19) United States
(12) Patent Application Publication
(10) Pub. No.: US 2005/0209216 A1
(43) Pub. Date: Sep. 22, 2005

(54) Phenyl-and pyridyl-diazaheterocycles having a tnf-modulating activity

(76) Inventors: Marco Baroni, Vanzago (IT); Bernard Bourrie, Saint-Gely-Du Fesc (FR); Pierre Casellas, Montpellier (FR)

Correspondence Address: ROSS J. OEHLER;AVENTIS PHARMACEUTICALS INC., ROUTE 202-206, MAIL CODE: D303A, BRIDGEWATER, NJ, 08807, US

(21) Appl. No.: 10/518,652

(22) PCT Filed: Jun. 16, 2003
(86) PCT No.: PCT/FR03/01813

§ 371 Date: Feb. 25, 2005

(30) Foreign Application Priority Data
Jun. 18, 2002 (FR) 02/07507

(51) Int. Cl. A61K 31/551; A61K 31/496; A61K 31/495; C07D 43/02; C07D 487/04

(52) U.S. Cl. 514/218; 514/248; 514/253.01; 514/255.03; 540/575; 544/360; 544/349

(57) ABSTRACT

The present invention relates to compounds of formula (I): 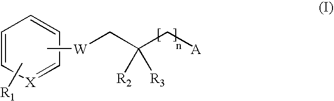
in which X represents N or CH; R₁ represents a hydrogen or halogen atom or a CF₃ group; W represents a diazoheterocycle; R₂ and R₃ independently represent a hydrogen atom or a methyl group; n is 0 or 1; A represents a quinoline or an isoquinoline which are optionally substituted.

The present invention relates to novel phenyl- and pyridyl-diazaheterocycles having a TNF modulating activity, the pharmaceutical compositions containing them and a method for their preparation.

U.S. Pat. No. 3,188,313 describes piperazines which are substituted with an indolylalkyl radical showing activity on the central nervous system, on the cardiovascular system and on the muscle and bone systems.

WO 01/29026 describes certain tetrahydropyridines substituted with a quinolinylalkyl or isoquinolylalkyl radical having activity on the modulation of TNF-alpha (Tumour Necrosis Factor).

TNF-alpha is a cytokine which has recently aroused interest as a mediator of immunity, of inflammation, of cell proliferation, of fibrosis, etc. This mediator is present in abundance in inflamed synovial tissue and exerts an important role in the pathogenesis of autoimmunity (Annu. Rep. Med. Chem., 1997, 32:241-250).

It has now been found that diazaheterocycles bearing a quinolinylalkyl or isoquinolylalkyl radical possess a potent activity toward the modulation of TNF-alpha.

Thus, the present invention relates, according to one of its aspects, to diazaheterocycles of formula (I): 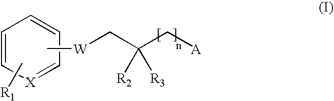
in which
X represents N or CH;
R₁ represents a hydrogen or halogen atom or a CF₃ group;
R₂ and R₃ independently represent a hydrogen atom or a methyl group;
n is 0 or 1;
W represents a diazoheterocycle of formula (a) to (d) 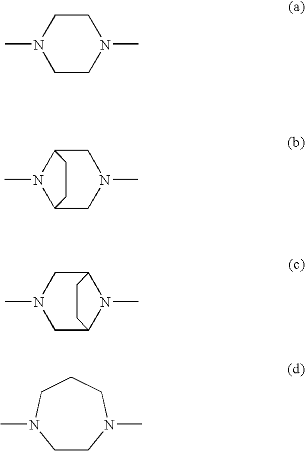
A represents a group of formula (e) or (f) 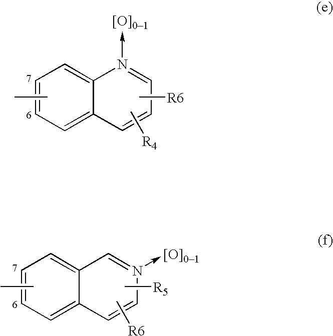
   where
R₄ represents a hydrogen or halogen atom, a (C₁-C₄)alkyl group, a CF₃ group, an amino, a mono(C₁-C₄)alkylamino or a di(C₁-C₄)alkylamino group;
R₅ represents a hydrogen or halogen atom, a (C₁-C₄)alkoxy group, a (C₁-C₄)alkyl group or a CF₃ group;
R₆ represents a hydrogen atom, a (C₁-C₄)alkyl group or a (C₁-C₄)alkoxy group;
   it being possible for only one or both of the atoms of the diazoheterocycles (a) to (d) to be oxidized; and their salts or solvates.

In the present description, the term “(C₁-C₄)alkyl” denotes a monovalent radical of a saturated straight-chain or branched-chain (C₁-C₄) hydrocarbon.

In the present description, the term “halogen” denotes an atom chosen from chlorine, bromine, iodine and fluorine.

As indicated in the formulae (e) and (f) above, the quinoline and isoquinoline rings may be attached to the remainder of the molecule of formula (I) by any one of the carbon atoms at the 6- or 7-position.

Preferred compounds of formula (I) are those where n is zero.

Other preferred compounds are those where R₂ and R₃ are each a hydrogen atom.

Other preferred compounds are those where R₁ is a CF₃ group.

Other preferred compounds are those where R₁ is a fluorine or chlorine atom.

Other preferred compounds are those where X is CH and R₁ is at the 2- or 3-position of the benzene.

Other preferred compounds are those where X is CH and R₁ is a CF₃ group.

Other preferred compounds are those where X is N and the pyridine is substituted at the 2,6-positions.

According to the present invention, the compounds of formula (I) can exist as N-oxide derivatives. As indicated above, the compounds of formula (I) can in particular bear one or two N-oxide groups on the diazoheterocycles (a) to (d) and/or an N-oxide group on the quinoline or isoquinoline of the group A. Although in principle the above three nitrogens can all be oxidized, the compounds bearing only one or two N-oxide groups, one on the diazoheterocycle and the other on the quinoline or isoquinoline, are preferred.

The salts of the compounds of formula (I) according to the present invention comprise both the addition salts with pharmaceutically acceptable inorganic or organic acids such as the hydrochloride, hydrobromide, sulphate, hydrogen sulphate, dihydrogen phosphate, citrate, maleate, tartrate, fumarate, gluconate, methanesulphonate, 2-naphthalenesulphonate, etc., and the addition salts which allow a suitable separation or crystallization of the compounds of formula (I), such as the picrate or oxalate, or the addition salts with optically active acids, for example camphorsulphonic acids and mandelic acids or substituted mandelic acids.

The optically pure stereoisomers, and the mixtures of isomers of the compounds of formula (I) due to the asymmetric carbon, when either one of R₂ or R₃ is a methyl and the other is a hydrogen, in any proportion, form part of the present invention.

The compounds of formula (I) can be synthesized by a method which involves a condensation/reduction reaction starting with a compound of formula (II): 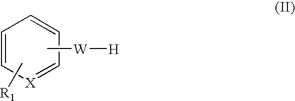
in which X, W and R₁ are as defined above, with an aldehyde of formula (III): 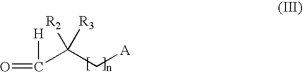
in which R₂, R₃, n and A are as defined above, the isolation of the compound of formula (I) and the optional conversion to one of its salts or solvates or to its N-oxide derivatives.

The condensation/reduction reaction is carried out by mixing the starting compounds (II) and (III) in an organic solvent such as an alcohol such as, for example, methanol, in an acidic medium, in the presence of a reducing agent such as sodium cyanoborohydride, according to conventional methods.

Alternatively, the compounds of formula (I) can also be prepared by a condensation which involves reacting a compound of formula (II) above with a compound of formula (IV) 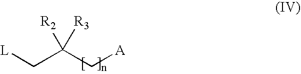
in which R₂, R₃, n and A are as defined above and L is a leaving group, isolating the compound of formula (I) and optionally converting to one of its salts or solvates or to its N-oxide derivatives.

The condensation reaction is normally carried out by mixing the starting compounds (II) and (IV) in an inert organic solvent, according to conventional methods.

The expression “inert organic solvent” is understood to mean a solvent which does not interfere with the reaction. Such solvents are for example alcohols such as methanol, ethanol, isopropanol or butanol.

As leaving group L, it is possible to use for example a chlorine or bromine atom or a mesyloxy (CH₃—SO₂—O—) group.

The reaction is carried out at a temperature of between −10° C. and the reflux temperature of the reaction mixture, the reflux temperature being preferred.

The reaction can be suitably carried out in the presence of a proton acceptor, for example an alkali metal carbonate or a tertiary amine such as triethylamine.

The reaction is normally stopped after a few hours, normally from 1 to 6 hours are sufficient to complete the condensation.

The desired compound of formula (I) is isolated according to conventional techniques in the form of a free base or of one of its salts. The free base may be converted to one of its salts by mere salification in an organic solvent such as an alcohol, preferably ethanol or isopropanol, an ether such as 1,2-dimethoxyethane, ethyl acetate, acetone or a hydrocarbon such as hexane.

The starting compounds of formula (II) containing a dinitrogen-containing ring (a) or (d) are known or they can be prepared in a similar manner to known compounds.

The starting compounds of formula (II) where the dinitrogen-containing ring is (b) or (c) and X is N are, for their part, also known or they can be prepared in a similar manner to known compounds, as described for example in J. Med. Chem., 1998, 41, 674-681.

The starting compounds of formula (II) where the dinitrogen-containing ring is (b) or (c) and X is CH can be prepared by the reaction of a bromobenzene optionally substituted with the ring (b) or (c), the nitrogen which should not take part in the reaction being suitably protected beforehand. Examples of such a reaction are given in the experimental section.

The compounds of formula (II′) 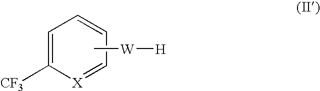
in which W represents a group of formula (b) or (c) above are novel and represent another aspect of the present invention.

The compounds of formulae (III) and (IV) are known and can be prepared in a similar manner to known compounds, for example as described in WO 01/29026.

The compounds of formula (I) bearing an N-oxide group on the nitrogen atom of the quinoline or isoquinoline can be prepared from the N-oxide derivatives of the compounds of formula (III).

The compounds of formula (I) bearing an N-oxide group on the nitrogen atoms of the rings (a) to (d) can be prepared by oxidation of the corresponding compound of formula (I). In this case, the compound of formula (I) as obtained by the above syntheses is subjected to an oxidation reaction according to the conventional methods, for example to a reaction with m-chloroperbenzoic acid in a suitable solvent, and isolated according to the usual techniques that are well known to those skilled in the art.

The compounds of the invention have advantageous properties with respect to the inhibition of TNF-α.

These properties were demonstrated with the aid of a test aimed at measuring the effect of molecules on the synthesis of TNF-α induced in Balb/c mice by lipopolysaccharide (LPS) from Escherichia Coli (055:B5, Sigma, St. Louis, Mo.).

The test products are administered orally to groups of 5 female 7- to 8-week old Balb/c mice (Charles River, France). One hour later, the LPS is administered intravenously (10 μg/mouse). The blood of each animal is taken 1.5 hours after the administration of the LPS. The samples are centrifuged and the plasma is recovered and frozen at −80° C. The TNF-α is measured using commercial kits (R and D, Abingdon, UK).

In this test, representative compounds of the invention were found to be very active, by inhibiting the synthesis of TNF-α even at very low doses.

By virtue of this activity and their low toxicity, the compounds of formula (I) and their salts or solvates can be used in the treatment of diseases linked to immune and inflammatory disorders or as analgesics. In particular, the compounds of formula (I) can be used for treating atherosclerosis, autoimmune diseases, diseases entailing demyelinization of the neurons (such as multiple sclerosis), asthma, rheumatoid arthritis, fibrotic diseases, pulmonary idiopathic fibrosis, cystic fibrosis, glumerulonephritis, rheumatoid spondylitis, osteoarthritis, gout, bone and cartilage resorbtion, osteoporosis, Paget's disease, multiple myeloma, uveoretinitis, septic shock, septicaemia, endotoxic shock, graft-versus-host reaction, graft rejection, adult respiratory distress syndrome, silicosis, asbestosis, pulmonary sarcoidosis, Crohn's disease, ulcerative colitis, amyotrophic lateral sclerosis, Alzheimer's disease, Parkinson's disease, disseminated lupus erythematosus, haemodynamic shock, ischaemic pathologies (myocardial infarction, myocardial ischaemia, coronary vasospasm, angina pectoris, cardiac insufficiency, heart attack), post-ischaemic reinfusion attacks, malaria, mycobacterial infections, meningitis, leprosy, viral infections (HIV, cytomegalovirus, herpesvirus), opportunistic infections associated with AIDS, tuberculosis, psoriasis, atopic dermatitis and contact dermatitis, diabetes, cachexia, cancer and radiation-mediated damage.

The compounds of formula (I) and the pharmaceutically acceptable salts and solvates thereof are preferably administered orally.

In the pharmaceutical compositions of the present invention for oral use, the active principle can be administered in unit administration forms, as a mixture with conventional pharmaceutical supports, to animals and human beings for the treatment of the abovementioned complaints. The appropriate unit administration forms comprise, for example, tablets, which may be splittable, gel capsules, powders, granules and oral solutions or suspensions.

When a solid composition in the form of tablets is prepared, the main active ingredient is mixed with a pharmaceutical vehicle such as gelatin, starch, lactose, magnesium stearate, talc, gum arabic or the like. The tablets can be coated with sucrose or other suitable materials or alternatively they can be treated such that they have sustained or delayed activity and such that they release a predetermined amount of active principle continuously.

A preparation in the form of gel capsules is obtained by mixing the active ingredient with a diluent and pouring the mixture obtained into soft or hard gel capsules.

A preparation in the form of syrup or elixir can contain the active ingredient together with a sweetener, preferably a calorie-free sweetener, methylparaben and propyl paraben as antiseptic agents, as well as a flavouring and a suitable colorant.

The water-dispersible powders or granules can contain the active ingredient as a mixture with dispersants or wetting agents, or suspending agents, such as polyvinylpyrrolidone, as well as with sweeteners or flavour enhancers.

The active principle can also be formulated in the form of microcapsules, optionally with one or more supports or additives.

In the pharmaceutical compositions according to the present invention, the active principle can also be in the form of an inclusion complex in cyclodextrins, or ethers or esters thereof.

The amount of active principle to be administered depends, as always, on the degree of progress of the disease as well as the age and weight of the patient. Nevertheless, the unit doses generally comprise from 0.001 mg to 100 mg, better still from 0.01 mg to 50 mg and preferably from 0.1 mg to 20 mg of active principle, advantageously from 0.5 mg to 10 mg.

According to another of its aspects, the present invention relates to a combination comprising a compound of formula (I) or one of its pharmaceutically acceptable salts or solvates, and at least one compound chosen from immunosuppressants, such as interferon beta-1b; adrenocorticotropic hormone; glucocorticoids such as prednisone or methylprednisolone; interleukin-1 inhibitors.

More particularly, the compounds of the invention can be combined with a compound chosen from roquinimex(1,2-dihydro-4-hydroxy-N,1-dimethyl-2-oxo-3-quinolinecarboxanilide), myloran (product from the company Autoimmune containing bovine myelin), antegren (monoclonal human antibody from the companies Elan/Athena Neurosciences) and recombinant interferon beta-1b.

Other possible combinations are those consisting of a compound of formula (I), or one of its pharmaceutically acceptable salts or solvates, and a potassium-channel blocker such as, for example, fampridine(4-aminopyridine).

According to another of its aspects, the invention relates to a method for treating diseases linked to immune and inflammatory disorders as well as in the treatment of pain, in particular atherosclerosis, autoimmune diseases, diseases entailing demyelinization of the neurons (such as multiple sclerosis), asthma, rheumatoid arthritis, fibrotic diseases, pulmonary idiopathic fibrosis, cystic fibrosis, glumerulonephritis, rheumatoid spondylitis, osteoarthritis, gout, bone and cartilage resorbtion, osteoporosis, Paget's disease, multiple myeloma, uveoretinitis, septic shock, septicaemia, endotoxic shock, graft-versus-host reaction, graft rejection, adult respiratory distress syndrome, silicosis, asbestosis, pulmonary sarcoidosis, Crohn's disease, ulcerative colitis, amyotrophic lateral sclerosis, Alzheimer's disease, Parkinson's disease, disseminated lupus erythematosus, haemodynamic shock, ischaemic pathologies (myocardial infarction, myocardial ischaemia, coronary vasospasm, angina pectoris, cardiac insufficiency, heart attack), post-ischaemic reinfusion attacks, malaria, mycobacterial infections, meningitis, leprosy, viral infections (HIV, cytomegalovirus, herpesvirus), opportunistic infections associated with AIDS, tuberculosis, psoriasis, atopic dermatitis and contact dermatitis, diabetes, cachexia, cancer and radiation-mediated damage, comprising the administration of a compound of formula (I) or a pharmaceutically acceptable salt or solvate thereof, alone or in combination with other active . principles.

The examples which follow illustrate the invention.

Preparation 1

### 8-(3-Trifluoromethylphenyl)-3,8-diazabicyclo[3.2.1]-octane and its Monohydrochloride Salt

### (i) 3-Benzyl-8-(3-trifluoromethylphenyl)-3,8-diazabicyclo[3.2.1]octane

640 mg (3.2 mmol) of 3-benzyl-3,8-diazabicyclo[3.2.1]octane are dissolved in 8 ml of anhydrous tetrahydrofuran and the medium is cooled to 0° C. under a nitrogen stream. 2 ml (3.2 mmol) of a butyllithium solution in hexane are carefully added to the mixture and the solution is allowed to acquire a deep red colour. 748 mg (3.96 mmol) of 3-trifluoromethyl-1-bromobenzene in 2 ml of anhydrous tetrahydrofuran are then added dropwise and the medium is stirred at 0° C. for 2 hours. The mixture is washed with water, the organic phase is dried over sodium sulphate and the solvent is evaporated under reduced pressure. An oil is obtained which is purified by chromatography on a silica gel column, eluting with an ethyl acetate/hexane 1/10 mixture. The title compound is obtained.

### (ii) 8-(3-Trifluoromethylphenyl)-3,8-diazabicyclo-[3.2.1]octane and its Monohydrochloride Salt

A solution of 800 mg (2.3 mmol) of the product of the preceding step and 100 mg of 10% Pd/C in 25 ml of anhydrous tetrahydrofuran and 0.5 ml of concentrated hydrochloric acid is hydrogenated at atmospheric pressure, at 47.5° C. The catalyst is filtered off, the solvent is evaporated under reduced pressure and the title compound is thus obtained in the form of a hydrochloride salt. m.p. 206-207° C.

Preparation 2

### 3-(3-Trifluoromethylphenyl)-3,8-diazabicyclo[3.2.1]-octane and its Monohydrochloride Salt

### (i) 8-Benzyl-3-(3-trifluoromethylphenyl)-3,8-diazabicyclo[3.2.1]octane

1.89 g (10 mmol) of 3-trifluoromethyl-1-bromobenzene, 2.32 g (11.5 mmol) of 8-benzyl-3,8-diazabicyclo[3.2.1]octane are dissolved in 40 ml of anhydrous toluene and 22.5 mg of palladium acetate, 93 mg (0.15 mmol) of BINAP (2,2′-bis(diphenylphosphino)-1,1′-binaphthyl) and a solution of potassium tert-butoxide in 18 ml of tetrahydrofuran are added thereto. The solution takes on a deep red colour and the reaction is allowed to proceed at 80° C. for 10 hours and then overnight at room temperature. The mixture is washed with water, extracted with ethyl acetate, the organic phase dried over sodium sulphate and the solvent evaporated under reduced pressure. An oil is obtained which is purified by chromatography on a silica gel column, eluting with an ethyl acetate/hexane 1/7 mixture. The title compound is obtained (Rf=0.31).

### (ii) 3-(3-Trifluoromethylphenyl)-3,8-diazabicyclo-[3.2.1]octane and its Monohydrochloride Salt

The compound of the preceding step is hydrogenated as described in Preparation 1(ii) and the title compound is thus obtained. m.p. 234-236° C.

### EXAMPLE 1

### 7-(2-(4-(3-Trifluoromethylphenyl)piperazin-1-yl)ethyl)isoquinoline and its Dihydrochloride Trihydrate

0.4 ml of 1-(3-trifluoromethylphenyl)piperazine (commercial product), 5 ml of methanol, 0.35 ml of glacial acetic acid and 0.18 g of sodium acetate are mixed. The medium is cooled to 0-5° C. and 0.38 g (0.0022 mol) of 7-isoquinolylacetaldehyde (as obtained in Preparation 1 of WO 01/29026) and, with care, 0.35 g of sodium cyanoborohydride are added thereto. The medium is stirred for 1 hour at 0-5° C. and then overnight at room temperature. 5 ml of concentrated hydrochloric acid are added, the medium is stirred for 10 minutes, the solvent evaporated under reduced pressure and the residue taken up in an ethyl acetate/dilute NH₄OH mixture. The two phases are separated, the organic phase is dried over sodium sulphate, filtered and the solvent is evaporated off. The residue is purified on a silica gel column, eluting with ethyl acetate. The title compound is obtained in the form of a base. The hydrochloride is prepared using a solution of isopropanol saturated with hydrochloric acid. 0.06 g of the title product is obtained. m.p. (dihydrochloride trihydrate) 210-212° C.

### EXAMPLE 2

### 7-(2-(4-(3-Trifluoromethylphenyl)piperazin-1-yl)ethyl)quinoline and its Hydrochloride

339 mg (1.78 mmol) of 7-(2-chloroethyl)-quinoline are dissolved in 12 ml of isopropanol and 791 mg (3.56 mmol) of 4-(3-trifluoromethylphenyl)-1,2,3,6-tetrahydropyridine are added thereto. The medium is heated under reflux for 4 hours and then stirred overnight at room temperature. The solvent is evaporated under reduced pressure and the crude product is obtained which is purified by chromatography on a silica gel column, eluting with ethyl acetate. The title product is thus obtained. Its dihydrochloride is prepared by reaction with hydrochloric acid in isopropanol.

m.p. 221-223° C.

### EXAMPLES 3 TO 13

The compounds of Examples 3 to 9 are prepared according to the procedures described in Example 1.

The compounds of Examples 10 to 13 are prepared according to the procedures described in Example 2.

The structures of the compounds and their characteristics are given in the following table.

TABLE

| Example | Structure | m.p. |
|---|---|---|
| 1 | 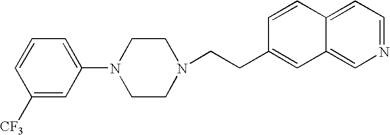 | 210-212° C. (dihydrochloride, trihydrate) |
| 2 | 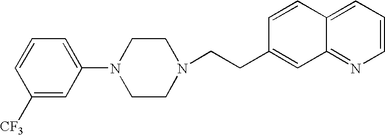 | 221-223° C. (dihydrochloride) |
| 3 | 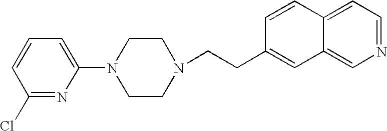 | 108-110° C. (dihydrochloride, dihydrate) |
| 4 | 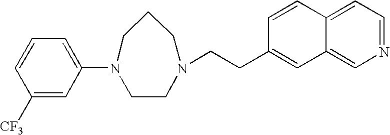 | 135-137° C. (oxalate) |
| 5 | 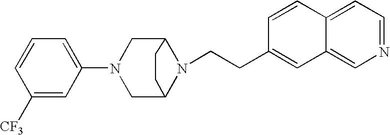 | 97-99° C. (oxalate) |
| 6 | 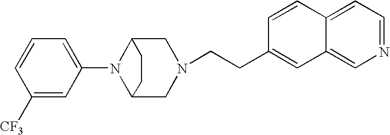 | 151-153° C. (dihydrochloride, dihydrate) |
| 7 | 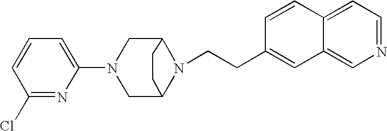 | 141-142° C. (base) |
| 8 | 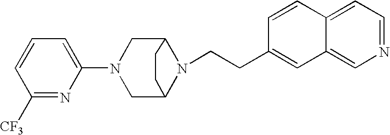 | 258-260° C. (dihydrochloride, dihydrate) |
| 9 | 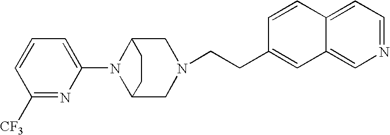 | 158-160° C. (dihydrochloride, dihydrate) |
| 10 | 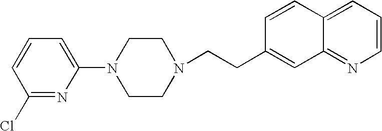 | 170-172° C. (oxalate) |
| 11 | 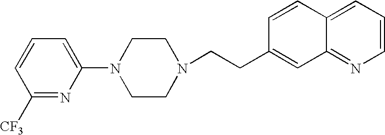 | 228-230° C. (dihydrochloride) |
| 12 | 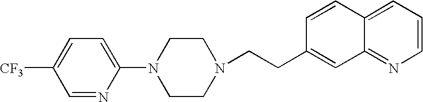 | 108-110° C. (dihydrochloride) |
| 13 | 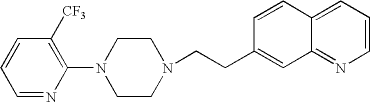 | 163-164° C. (dihydrochloride) |

1. Compound of formula (I): 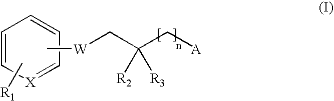
in which
X represents N or CH;
R₁ represents a hydrogen or halogen atom or a CF₃ group;
R₂ and R₃ independently represent a hydrogen atom or a methyl group;
n is 0 or 1;
W represents a diazoheterocycle of formula (a) to (d) 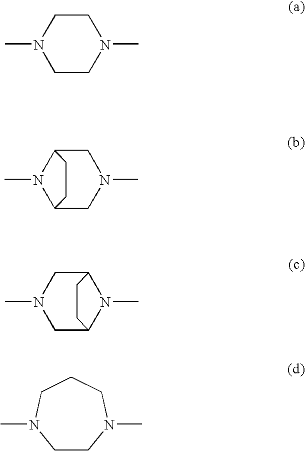
A represents a group of formula (e) or (f) 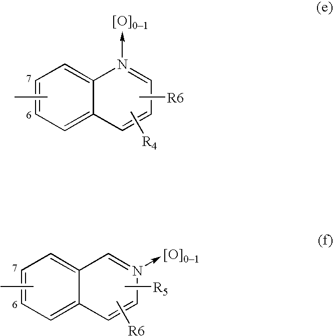
where
R₄ represents a hydrogen or halogen atom, a (C₁-C₄)alkyl group, a CF₃ group, an amino, a mono(C₁-C₄)alkylamino or a di(C₁-C₄)alkylamino group;
R₅ represents a hydrogen or halogen atom, a (C₁-C₄)alkoxy group, a (C₁-C₄)alkyl group or a CF₃ group;
R₆ represents a hydrogen atom, a (C₁-C₄)alkyl group or a (C₁-C₄)alkoxy group; it being possible for only one or both of the atoms of the rings (a) to (d) to be oxidized;
and their salts or solvates.

2. Compound according to claim 1, where n is zero.

3. Compound according to claim 1 wherein R₂ and R₃ are each a hydrogen atom.

4. Compound according to claim 1 wherein R₁ is a CF₃ group.

5. Compound according to claim 1 wherein R₁ is a fluorine or chlorine atom.

6. Compound according to claims 1 claim 1 wherein X is CH and R₁ is at the 3-position of the benzene.

7. Compound according to claim 1 wherein X is CH and R₁ is at the 2-position of the benzene.

8. Compound according to claim 1 wherein X is N and the pyridine is substituted at the 2,6-positions.

9. Compound according to claim 1 chosen from its mono-N-oxide derivatives, its bis-N-oxides and its tri-N-oxides.

10. A method for preparing the compound of claim 1; wherein there are carried out a condensation/reduction reaction of a compound of formula (II): 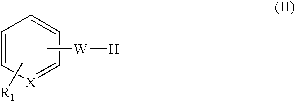
in which X, W and R₁ are as defined in claim 1, with an aldehyde of formula (III): 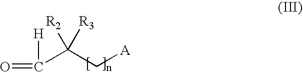
in which R₂, R₃, n and A are as defined above, the isolation of the compound of formula (I) and the optional conversion to one of its salts or solvates or to its N-oxide derivatives.

11. Compound of formula (II′) 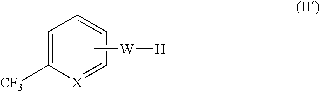
in which W represents a group of formula (b) or (c) according to claim 1, and its salts or solvates.

12. A pharmaceutical composition containing, as active ingredient, a compound of formula (I) according to claim 1 or one of its pharmaceutically acceptable salts or solvates.

13. Composition according to claim 12 wherein it contains from 0.001 to 100 mg of active ingredient.

14. A method for the treatment of diseases linked to immune and inflammatory disorders or for the treatment of pain which comprises administering to a patient in need thereof an effective amount of a compound according to claim 1.

15. (canceled)

16. Compound according to claim 2 wherein R₂ and R₃ are each a hydrogen atom.

17. Compound according to claim 2 wherein R₁ is a CF₃ group.

18. Compound according to claim 2 wherein R₁ is a fluorine or chlorine atom.

19. Compound according to claim 2 wherein X is CH and R₁ is at the 3-position of the benzene.

20. Compound according to claim 3 wherein X is CH and R₁ is at the 3-position of the benzene.

21. Compound according to claim 2 wherein X is CH and R₁ is at the 2-position of the benzene.

22. Compound according to claim 3 wherein X is CH and R₁ is at the 2-position of the benzene.

23. Compound according to claim 2 wherein X is N and the pyridine is substituted at the 2,6-positions.

24. Compound according to claim 3 wherein X is N and the pyridine is substituted at the 2,6-positions.

25. A pharmaceutical composition containing, as active ingredient, a compound of formula (I) according to claim 2 or one of its pharmaceutically acceptable salts or solvates.

26. A pharmaceutical composition containing, as active ingredient, a compound of formula (I) according to claim 3 or one of its pharmaceutically acceptable salts or solvates.

27. A pharmaceutical composition containing, as active ingredient, a compound of formula (I) according to claim 4 or one of its pharmaceutically acceptable salts or solvates.

28. A pharmaceutical composition containing, as active ingredient, a compound of formula (I) according to claim 5 or one of its pharmaceutically acceptable salts or solvates.

29. A pharmaceutical composition containing, as active ingredient, a compound of formula (I) according to claim 6 or one of its pharmaceutically acceptable salts or solvates.

30. A pharmaceutical composition containing, as active ingredient, a compound of formula (I) according to claim 7 or one of its pharmaceutically acceptable salts or solvates.

31. A pharmaceutical composition containing, as active ingredient, a compound of formula (I) according to claim 8 or one of its pharmaceutically acceptable salts or solvates.

32. A pharmaceutical composition containing, as active ingredient, a compound of formula (I) according to claim 9 or one of its pharmaceutically acceptable salts or solvates.

33. A method for the treatment of diseases linked to immune and inflammatory disorders or for the treatment of pain which comprises administering to a patient in need thereof an effective amount of a compound according to claim 2.

34. A method for the treatment of diseases linked to immune and inflammatory disorders or for the treatment of pain which comprises administering to a patient in need thereof an effective amount of a compound according to claim 3.

35. A method for the treatment of diseases linked to immune and inflammatory disorders or for the treatment of pain which comprises administering to a patient in need thereof an effective amount of a compound according to claim 4.

36. A method for the treatment of diseases linked to immune and inflammatory disorders or for the treatment of pain which comprises administering to a patient in need thereof an effective amount of a compound according to claim 5.

37. A method for the treatment of diseases linked to immune and inflammatory disorders or for the treatment of pain which comprises administering to a patient in need thereof an effective amount of a compound according to claim 6.

38. A method for the treatment of diseases linked to immune and inflammatory disorders or for the treatment of pain which comprises administering to a patient in need thereof an effective amount of a compound according to claim 7.

39. A method for the treatment of diseases linked to immune and inflammatory disorders or for the treatment of pain which comprises administering to a patient in need thereof an effective amount of a compound according to claim 8.

40. A method for the treatment of diseases linked to immune and inflammatory disorders or for the treatment of pain which comprises administering to a patient in need thereof an effective amount of a compound according to claim 9.
